(19) United States
(12) Patent Application Publication
(10) Pub. No.: US 2024/0203599 A1
(43) Pub. Date: Jun. 20, 2024

(54) METHOD AND SYSTEM OF FOR PREDICTING DISEASE RISK BASED ON MULTIMODAL FUSION

(71) Applicant: SHANDONG UNIVERSITY, Jinan, Shandong (CN)

(72) Inventors: Zhi LIU, Jinan (CN); Yujun LI, Jinan (CN); Xifeng HU, Jinan (CN); Weifeng HU, Jinan (CN)

(73) Assignee: SHANDONG UNIVERSITY, Jinan, Shandong (CN)

(21) Appl. No.: 17/910,556

(22) PCT Filed: Jul. 16, 2021
(86) PCT No.: PCT/CN2021/106860

§ 371 Date: Sep. 9, 2022

(30) Foreign Application Priority Data
Apr. 30, 2021 (CN) 202110486200.2

(51) Int. Cl. G16H 50/30; G16H 10/60

(52) U.S. Cl. CPC G16H 50/30; G16H 10/60

(57) ABSTRACT

A method and system of predicting disease risk based on multimodal fusion, the method comprises: obtaining electronic health record (EHR) data of the patient, inputting the EHR data into the disease risk prediction model to obtain the disease risk prediction result; and outputting the disease risk prediction result; wherein, the disease risk prediction model performing steps of: identifying the EHR data as the structured data and the unstructured data; performing the data cleaning on the structure data and the unstructured data; extracting structured data features and unstructured data features; extracting fusion features, wherein the fusion features are features fusing the unstructured data feature and the structured data feature; and, performing the disease risk prediction on the fusion features.

### CROSS-REFERENCES TO RELATED APPLICATIONS

This application claims priority benefits to Chinese Patent Application No. 202110486200.2, filed 30 Apr. 2021, the contents of which are incorporated herein by reference.

### TECHNICAL FIELD

The present invention relates to the field of medical big data information processing, and in particular to a method and system of predicting disease risk based on multimodal fusion.

### BACKGROUND

The disclosure of the information in the background technical part is only to increase the understanding of the overall background of the application and is not necessarily deemed to recognize or imply in any form that the information constitutes the prior art that has become well known to those skilled in the art.

Electronic health record (EHR) creates a vast amount of inexpensive data for health research, including electronic medical records, past medical history information, textual records of patient medical records, and so on. Digitization and subsequent analysis of medical records constitute a field of digital transformation to collect a variety of medical information about patients in the form of the EHR, including digital measurements (lab results), verbal descriptions (symptoms and notes, vital signs, etc.), images (X-rays, CT, MR scans, etc.), and to document the patient's treatment. This digitization creates opportunities to mine health records to improve the quality of care and clinical outcomes.

However, clinicians have limited time to process all available data and detect patterns in similar medical records. The EHR contains structured and unstructured data with important research and clinical value. With the standardization and digitalization of a large number of EHR data, it is urgent to realize personalized medical treatment by mining a large number of multi-source heterogeneous data and then establishing risk prediction models. Most previous attempts were based on structured EHR fields, and a large amount of information in unstructured text data was lost.

### SUMMARY

Based on understanding the shortcomings of the prior art, the inventor finds that the limitation and one-sidedness caused by single data can be avoided by effectively mining the medical text and deeply fusing and researching the multi-source heterogeneous data through an effective data fusing means. Therefore, the inventor is further combining deep learning with disease prediction for research. However, the combination of the two is accompanied by the following problems, including:
problems with uneven number and distribution of datasets: data collection without purpose often results in the inability to form a systematic system for the completeness, accuracy, and granularity of recorded data, resulting in missing and unstandardized data. Therefore, a certain amount of human and material resources are required for data collection. Limited by time and financial resources, the number of good samples that can be obtained is limited, for example, in some embodiments of the present invention, the number of good samples obtained is only 1300, and the distribution of positive and negative samples is unbalanced, which will greatly affect the learning and training of the deep neural network.

Problems that medical text data cannot be directly used for computation: in the existing processing mode, the medical text usually needs to be digitally represented first. However, these text data are usually long text with medical entities, and the vector representation of medical text data by a convolutional neural network (CNN), word2vec (word vector generation model), long-short term memory (LSTM), bi-directional long-short term memory (Bi-LSTM), etc. is not satisfactory.

As well, most of the current real clinical data are in the form of multimodality. However, there is less research on multimodality now, and a lot has been done with single-point breakthroughs, only considering single unimodal factors cannot comprehensively evaluate potential risks, and clinical data are not fully exploited and utilized.

For solving the defects in existing studies and the above-mentioned problems, according to the present invention, an effective vectorized representation of textual medical records is performed by a stacked Transformer encoder module, which effectively captures the rich semantic relationships contained in the anterior-posterior sequence in long texts and provides a correct representation of medical entities. Then, a feature-level fusion of heterogeneous data from multiple sources is performed to fully take into account the characteristics of different modal data, which in turn leads to the prediction of patient outcomes. The present invention provides a method for processing EHR data (including structured data and unstructured data) and constructs a disease risk prediction model based on multimodal fusion, provides a method and system for making predictions using the model, and a software device for implementing these functions, etc. The present invention improves the prognosis of the patient's outcome by fully integrating and mining the demographic information, the treatment information, the diagnostic information, the laboratory information, and the relevant text treatment medical records of the patient, which can effectively help the physician provide effective reference information, prognosis the development of the patient's condition, assist the physician in formulating a corresponding treatment plan, help save the patient in time and prevent the condition from developing in the direction of deterioration. At the same time, it can show patients the direction of disease development after personalized treatment at each clinical visit to improve their motivation for treatment.

Multimodal data refers to data collected in a variety of different devices or scenarios. Real-world datasets tend to be multimodal, such as a story can be narrated by text as well as by images or audio; a document can be represented in a plurality of different languages and can be represented by user ratings, etc. Multimodal databases are built to analyze and process multimodal data to obtain important features and representative search tags and use them as a basis to build a database for subsequent data retrieval.

Unstructured data refers to the data without fixed structure, for example, office documents in all formats, text, pictures, all kinds of reports, images, audio, and video type information. Unstructured data in medicine includes medical images, electrocardiograms, text medical records, etc.

Structured data: traditional relational data model, row data, data stored in the database and represented by two-dimensional table structure, for example, data stored in CSV and Excel, and two-dimensional table.

Specifically, the present invention provides the following technical features, and the combination of one or more of the following technical features constitutes the technical solution of the present invention.

In a first aspect of the present invention, the present invention provides a method of predicting disease risk based on multimodal fusion, the method comprises:
obtaining EHR data of a patient to be predicted, the EHR data comprising structured data and unstructured data; in an embodiment of the present invention, the unstructured data refers in particular to text;
inputting the EHR data into a disease risk prediction model to obtain a disease risk prediction result; and
outputting the disease risk prediction result.

Wherein, the disease risk prediction model performs the steps:
extracting structured data features and unstructured data features;
fusing the structured data features and the unstructured data features and extracting fusion features; and
making decisions on the fusion features to obtain the disease risk prediction results.

In some embodiments of the present invention, the prediction model of disease risk further comprises the step of performing a data cleaning before extracting the structured data features and the unstructured data features;
wherein, the data cleaning includes a replacement of outlier values, completing missing values using mean values, and removing dirty read.

In some embodiments of the present invention, a Fully Convolutional Network (FCN) is used to extract structured data features.

In some embodiments of the present invention, a Bidirectional Encoder Representation from Transformers (BERT) is used to extract unstructured features.

In some embodiments of the present invention, an operation of extracting the fusion features comprises: connecting the unstructured data features and the structured data features in parallel along a specified dimension, reducing an imbalance rate through a method of analyzing minority class sample data and newly generating a sample of the class by using a Synthetic Minority Oversampling Technique (SMOTE), then extracting the fusion features by using a piecewise pooling operation.

In some embodiments of the present invention, when the prediction is performed, inputting the fusion features as inputs to fully connected dence layers, and then performing the disease risk prediction by a Softmax classifier.

As well as, in the embodiments of the present invention, the present invention adopts a weighting of cross-entropy loss and hinge loss to jointly constrain the model. The cross-entropy loss can measure the difference degree between two different probability distributions in the same random variable, and the smaller the value of the cross-entropy loss value, the closer the two probability distributions are. However, using the cross-entropy loss alone tends to lead to confusion in the classification of boundary variables, and the hinge loss is specially used for binary classification problems, which requires not only correct classification, but also high certainty that the loss will be as small as possible. Since the hinge loss not only measures how well the model fits the training data but also measures the complexity of the model itself by adding a regularization term, it can greatly reduce the fitting risk.

In a second aspect of the present invention, the present invention provides a method for processing EHR data, comprising:
obtaining EHR data, the EHR data comprising structured data and unstructured data;
performing data processing on structured data and unstructured data separately, including performing data cleaning to obtain cleaned structured data and cleaned unstructured data, performing feature extraction to obtain unstructured data features and structured data features, fusing unstructured data features and structured data features, and extracting fused features; and
using the fused features as data to be identified for medical purposes.

In some embodiments of the present invention, the data cleaning comprises replacement of outlier values, complementation of missing values using mean values, and removal of dirty read; preferably, the unstructured data is text.

In some embodiments of the present invention, an FCN is used for extracting structured data features; a BERT is used for extracting unstructured features.

In some embodiments of the present invention, an operation of extracting the fusion features comprises: connecting the unstructured data features and the structured data features in parallel along a specified dimension, reducing an imbalance rate through a method of analyzing minority class sample data and newly generating the sample of the class by using a SMOTE, and then extracting fusion features by using a piecewise pooling operation.

In a third aspect of the present invention, a method of constructing a disease risk prediction model of the present invention, comprising:
obtaining EHR data of a patient with a known disease risk outcome, the data comprising structured data and unstructured data; building a dataset based on the EHR data, the dataset comprises a structured dataset and an unstructured dataset; and building a label set based on the known outcomes;
building a disease risk prediction network, comprising: building a structured data feature extraction module for extracting features of the structured data, an unstructured data feature extraction module for extracting features of the unstructured data, and a feature fusion module, then connecting the structured data feature extraction module and the feature extraction module unstructured data in parallel and then being connected in series with a decision layer of the feature fusion module; the disease risk prediction network is implemented bases on a Pytorch framework;
training the disease risk prediction network using the datasets (the structured dataset and the unstructured dataset) with the label set as a label to build the disease risk prediction model; and
adopting a weighting of a cross-entropy loss and a hinge loss to jointly constrain the model.

In some embodiments of the present invention, data cleaning is performed on EHR data before building a dataset, wherein the data cleaning comprises replacement of outlier values, complementation of missing values using mean values, and removal of dirty read.

In some embodiments of the present invention, the structured data feature extraction module is an FCN module, and the unstructured data feature extraction module is a BERT module (transformer module).

In some embodiments of the present invention, the feature fusion module performs the steps of connecting the unstructured data features and the structured data features in parallel along a specified dimension, reducing an imbalance rate through a method of analyzing minority class sample data and newly generating samples of the class by using a SMOTE and then extracting the fusion features by using a piecewise pooling operation.

In some embodiments of the present invention, when the dataset is used for training, the fusion features, as the input, are input to the fully connected dence layers to train the Softmax classifier.

In addition, the present invention further comprises the disease risk prediction model based on the multimodal fusion constructed by the third aspect of the present invention as mentioned above.

In a fourth aspect of the present invention, the present invention provides a disease risk prediction system based on multimodal fusion, comprising:
a feature extraction module, being used to perform feature extraction on EHR data to obtain unstructured data features and structured data features;
a feature fusion module, being used to fuse the unstructured data features and the structured data features to extract and obtain fusion features; and
a classification module, being used to obtain a disease risk prediction result using the fusion features as an input.

In some embodiments of the present invention, the feature extraction module comprises a structured data feature extraction module and an unstructured data feature extraction module;
wherein, the structured data feature extraction module uses structured data after pre-processing as an input of an FCN, maps the data to each hidden semantic node, and obtains the structured data features.
wherein, the unstructured data feature extraction module uses a BERT to perform the feature extraction on unstructured data after pre-processing; preferably, the BERT has a BERT Encoder consists of multiple BERT Layers, and each the BERT Layer is an Encoder Block in the Transformer; each the Encoder Block has two layers, which are a self-attentive mechanism layer and a feed-forward neural network layer, respectively.

In some embodiments of the present invention, the feature fusion module connects the unstructured data features and the structured data features in parallel along the specified dimension, uses a SMOTE to reduce the imbalance rate by analyzing minority class sample data and newly generating the sample of the class, and then extracts the fusion features by using a piecewise pooling operation.

In some embodiments of the present invention, the classification module inputs the fusion features as input to the fully connected dence layers, and then a Softmax classifier is used to classification to obtain a disease risk prediction result.

In some embodiments of the present invention, the system further comprises a data acquisition module for obtaining EHR data.

In some embodiments of the present invention, the system further comprises a data cleaning module for pre-processing the EHR data after obtaining the EHR data and before performing the feature extraction of the EHR data; wherein, the pre-processing comprises performing operations of replacing outlier values and completing missing values using mean values and removing dirty read using the EHR data cleaning module.

In some embodiments of the present invention, the system further comprises a result output module for outputting the disease risk prediction results.

In a fifth aspect of the present invention, the present invention provides a computer device comprising a memory and a processor, the memory storing a computer program; when the computer program being executed by the processor, implementing the steps of the method according to any one of the above first and/or second and/or third aspects of the present invention as mentioned above.

In a sixth aspect of the present invention, the invention provides a computer readable storage medium having stored thereon computer program instructions which, when executed by a processor, implement the steps of the method according to any one of the above first and/or second and/or third aspects of the present invention as mentioned above.

By one or more of the technical solutions described above, the following beneficial effects can be achieved:

The invention provides an end-to-end patient outcome prediction model, by automatically reading a patient's EHR data, using the read data as an input of the model, and after mining and analyzing the corresponding data by combining a deep learning method, and an output is a predicted event outcome of the patient. It can effectively help physicians provide effective reference information to predict the development of patients' conditions and give timely help and treatment. It also increases the motivation of patients to cooperate with treatment.

The invention adopts the bi-directional language model BERT for feature extraction of medical text, which can do parallel computation on multiple sets of inputs and capture different subspace information. The attention mechanism is introduced to help the model acquire the context information more effectively, learn the word dependency relationship inside the sentence and capture the internal structure of the sentence. The model is pre-trained with Chinese medical question and answer, Chinese medical encyclopedia, and Chinese electronic medical records, and medical entities such as “abdominal pain” can be more effectively vectorized.

The present invention adopts multimodal fusion technology to preprocess, analyze and mine data such as electronic medical records, past medical history information, text records of the patient's medical record, etc. to build a risk prediction model for predicting the outcome of the patient, providing an auxiliary means for the utilization of clinical real data and the evaluation of the disease outcome, helping physicians to provide personalized treatment plans for each patient.

### BRIEF DESCRIPTION OF THE DRAWINGS

The accompanying drawings constituting a part of the present invention are used to provide a further understanding of the present invention. The exemplary examples of the present invention and descriptions thereof are used to explain the present invention, and do not constitute an improper limitation of the present invention.

FIG. 1 is a flow chart of a method of processing EHR data in the first example of the present invention.

FIG. 2 is a structure diagram of a system for processing the EHR data in the first example of the present invention.

FIG. 3 is a functional flowchart of a feature fusion module in one or more examples of the present invention.

FIG. 4 is a flowchart of a method of predicting disease risk based on multimodal fusion in the third example of the present invention.

FIG. 5 is a functional flowchart of a prediction model of disease risk in one or more examples of the present invention.

FIG. 6 is a structure diagram of a system of predicting risk based on multimodal fusion in the fourth example of the present invention.

FIG. 7 is a structural diagram of the system of predicting risk based on multimodal fusion in the fourth example of the present invention.

FIG. 8 is a structural diagram of the system of predicting risk based on multimodal fusion in the fourth example of the present invention.

### DETAILED DESCRIPTION

The present application is further described below in connection with specific embodiments. It should be understood that these embodiments are intended to illustrate the present application only and are not intended to limit the scope of the present application.

The term “and/or,” as used herein, is merely an association that describes an association of objects, meaning that there can be three relationships, e.g., A and/or B, which can represent: there are three cases, namely, A exists alone, B exists alone, and A and B exist simultaneously. In this paper, the term “/and” describes another associative object relationship, indicating that there can be two relationships. For example, A/and B can indicate: In addition, the word “/” in this context generally indicates that the context object is an “or” relationship.

The terminology used herein is for the purpose of describing particular embodiments only and is not intended to be limiting of example embodiments of the present application. As used herein, the singular forms “a”, “an” and “the” are intended to include the plural forms as well, unless the context clearly indicates otherwise. It will be further understood that the terms “comprise”, “comprising”, “include,” and/or “including,” when used herein, specify the presence of stated features, integers, steps, operations, elements, and/or components, and do not preclude the presence or addition of one or more other features, integers, steps, operations, elements, components, and/or groups thereof.

It should be understood that although that term first, second, third, etc. may be adopted in the present application to describe various information, such information should not be limit to these terms. These terms are only used to distinguish the same type of information from each other. For example, first information may also be referred to as second information, and similarly, second information may also be referred to as first information, without departing from the scope of the present application. Depending on the context, the term “if” as used herein may be interpreted as “at the time of” or “when” or “in response to a determination”.

Furthermore, the particular features, structures, functions, or characteristics may be combined in any suitable manner in one or more embodiments. For example, a first example may be combined with a second example, so long as particular features, structures, functions, or characteristics associated with such examples or specific embodiments are not mutually exclusive.

In a first example of the present invention, the present invention provides a method for processing EHR data, comprising: obtaining EHR data, the EHR data comprising structured data and unstructured data;
processing the EHR data, and the processing process is shown in FIG. 1, comprising: performing data processing on structured data and unstructured data separately, including performing data cleaning to obtain cleaned structured data and cleaned unstructured data, performing feature extraction to obtain unstructured data features and structured data features, fusing unstructured data features and structured data features, and extracting fused features; and
using the fused features as data to be identified for medical purposes.

In addition, according to the method in the first example, the present invention further provides a system for processing EHR data, wherein, core modules thereof comprise a feature extraction module and a feature fusion module.

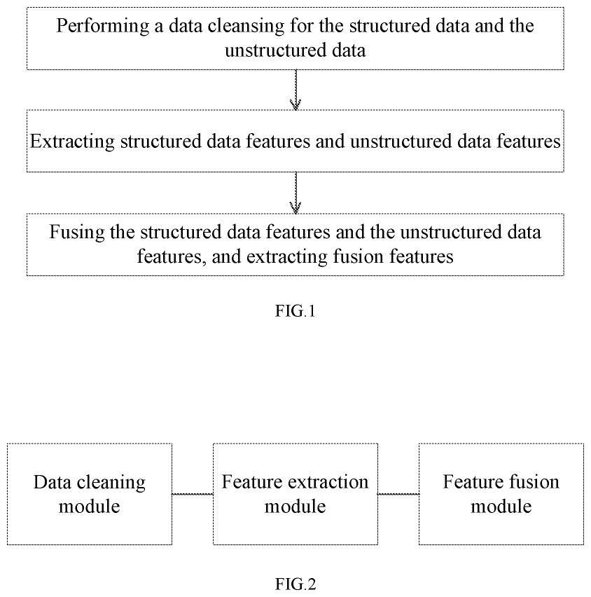

Optionally, after obtaining the EHR data to be processed, data cleaning can be performed on the data, so that the system further comprises a data cleaning module, as shown in FIG. 2.

Wherein, the data cleaning module performs operations of replacing outlier values and using a mean value to complete missing values and remove dirty reads. For example, the data can be filtered for the outlier values and replaced with null values, then the data can be weighted and averaged, and the outlier values and missing values can be replaced with the mean values, and Statistical Product Service Solutions (SPSS) can be used to clean the data.

The feature extraction module performs feature extraction on the structured data and unstructured data (such as text) contained in the EHR data; optionally, the feature extraction module comprises a structured data feature extraction module and an unstructured data feature extraction module.

Wherein, the structured data feature extraction module uses the cleaned structured data as the input of the FCN, maps the data to each hidden semantic node, and obtains the structured data features; in the present embodiment, the structured data feature extraction module learns the weights W through a Dence layer and then obtains reset features of the structured data, and due to a discrete nature of the data, the position information between the features has little impact on the decision making, so the position information can be optionally discarded in this process.

The unstructured data feature extraction module uses BERT to extract features from the cleaned unstructured text data. The BERT comprises BERT Encoder, the BERT Encoder comprises multiple layers of BERT Layer, each layer of the BERT Layer is an Encoder Block in Transformer; each encoder layer contains two layers, which are a self-attentive mechanism layer and a feed-forward neural network layer, respectively. In the present embodiment, for the module of unstructured text data mining, a stacked Transformer encoder module is used to obtain the word embedding tensor, sentence blocking tensor, and position encoding tensor to extract the semantic information, sentence information, and position information of the medical text data, respectively, to calculate the vectorized representation of the text medical record.

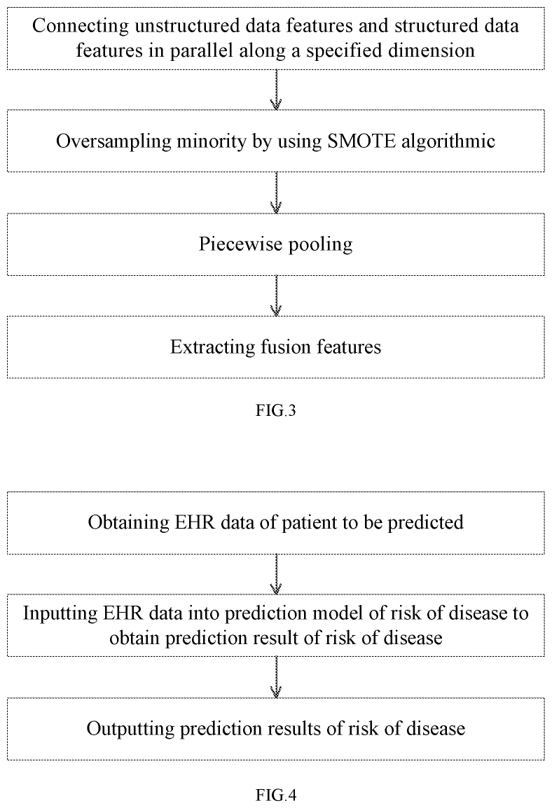

For the feature fusion module, as shown in FIG. 3, a connection layer connects the structured data features and the unstructured data features in parallel along the specified dimension, adopts the SMOTE to reduce the imbalance rate by analyzing the minority class sample data and newly generating the sample of the class, and extracts the important information of the data with different structures respectively according to the different data types by adding the piecewise pooling operation. Since medical data usually has a small sample size and batch normalization is affected by the size of batch_size, in the example, the output of each sub-layer is normalized using layer normalization.

In a second example of the present invention, the present invention provides a method for building a prediction model of disease risk, comprising.
obtaining EHR data of patients with known disease risk outcomes (the data comprises structured data and unstructured data, the unstructured data mainly refers to texts); building a dataset (structured dataset and text dataset) based on the EHR data, and building a label set based on known outcomes;
optionally, performing data cleaning on the obtained EHR data, the data cleaning comprises replacement of outlier values, complementation of missing values using mean values, and removal of dirty read;
building a disease risk prediction network, comprising: building a feature extraction module (FCN) for extracting the structured data, a feature extraction module (transformer module) for extracting the unstructured data, and a feature fusion module, then connecting the structured data feature extraction module and the feature extraction module unstructured data in parallel and then being connected with the feature fusion module in series at a decision layer; the disease risk prediction network is implemented on that basis of a Pytorch framework;
training the disease risk prediction network using the datasets (the structured dataset and the unstructured dataset) with the label set as a label to build the disease risk prediction model; in the example, the disease risk prediction model was constructed using the disease risk outcome as the label and the fusion features as the input to the fully connected layer and training a Softmax classifier;
inputting the EHR data of the patient to be predicted into the trained disease risk prediction model, then outputting the outcome attribute of the patient.

Furthermore, adopting a weighting of a cross-entropy loss and a hinge loss to jointly constrain the model. The cross-entropy loss can measure the difference degree between two different probability distributions in the same random variable, wherein, the smaller the cross-entropy loss value is, the closer the two probability distributions are. However, using the cross-entropy loss alone tends to lead to confusion in the classification of boundary variables, and the hinge loss is specially used for binary classification problems, which requires not only correct classification, but also high certainty that the loss will be as small as possible. Since the hinge loss not only measures how well the model fits the training data but also measures the complexity of the model itself by adding a regularization term, it can greatly reduce the fitting risk.

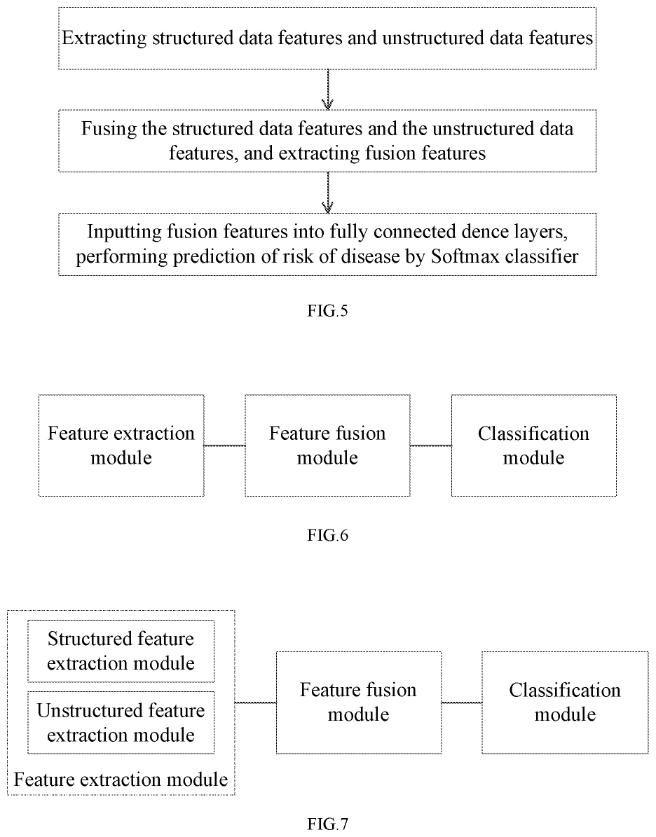

In a third example of the present invention, according to the disease risk prediction model constructed in the second example, the present invention provides a method for predicting disease risk based on multimodal fusion, as shown in FIG. 4, comprising:
obtaining EHR data of a patient to be predicted, and the EHR data comprises structured data and unstructured data (text);
inputting the obtained EHR data into a disease risk prediction model to obtain a disease risk prediction result; and
outputting the disease risk prediction result.

Wherein, steps performed by the disease risk prediction model, as shown in FIG. 5, comprise:
extracting structured data features and unstructured data features;
extracting fusion features, the fusion features being fusion features of the unstructured data features and the structured data features; and
decision-making on the fusion features to obtain the disease risk prediction result.

In the example, the disease risk prediction model is jointly constrained by a weighting of cross-entropy loss and hinge loss. The cross-entropy loss can measure the difference degree between two different probability distributions in the same random variable, wherein, the smaller the cross-entropy loss value is, the closer the two probability distributions are. However, using the cross-entropy loss alone tends to lead to confusion in the classification of boundary variables, and the hinge loss is specially used for binary classification problems, which requires not only correct classification, but also high certainty that the loss will be as small as possible. Since the hinge loss not only measures how well the model fits the training data but also measures the complexity of the model itself by adding a regularization term, it can greatly reduce the fitting risk.

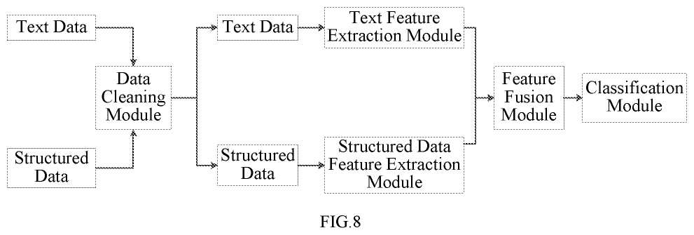

In a fourth example of the present invention, the present invention provides a disease risk prediction system based on multimodal fusion, as shown in FIG. 6, comprising: a feature extraction module, a feature fusion module, and a classification module.

Wherein, the feature extraction module comprises a structured data feature extraction module and an unstructured data feature extraction module, as shown in FIG. 7.

Based on the example, the disease risk prediction system based on multimodal fusion further comprises a data acquisition module and/or a data cleaning module, and/or a result output module.

For example, in the example, the system is as shown in FIG. 8.

As shown in FIG. 8, after the system obtains the EHR data (including the structured data and the unstructured data, such as text) of the patient to be predicted, the data cleaning module performs a pre-process on the EHR data, comprising a replacement of outlier values, complementation of missing values using mean values, and removal of dirty read.

The unstructured data such as text data after cleaning is subjected to feature extraction in a text feature extraction module, and the medical text data is subjected to feature extraction by applying a bidirectional language model BERT in the module. The core of the model has a BERT Encoder consists of multiple BERT Layers, and each the BERT Layer is an Encoder Block in the Transformer. Each the Encoder Block has two layers, which are a self-attentive mechanism layer and a feed-forward neural network layer.

The structured data after the cleaning process is subjected to feature extraction within the structured data feature extraction module, wherein, the structured data after the cleaning process is used as the input of FCN to map the original data to each hidden semantic node to obtain the structured data features.

As shown in FIG. 3, the fusion module splices the features of the structured data and the features of the text data in parallel along the specified dimension and uses the SMOTE to analyze the minority class sample data and newly generate the sample of the class to reduce the imbalance rate. Then a piecewise pooling operation is used to extract important information from different structured data to obtain fusion features.

The classification module takes the extracted fusion features after fusion as input to the fully connected layer and then predicts the outcome of the patient through the Softmax classifier.

Further, the prediction results obtained by the classification module are output by the result output module.

The physician can draw a conclusion based on the output combined with his or her judgment.

The system described in the example can implement the method for disease risk prediction based on multimodal fusion in the third example.

In a fifth example of the present invention, the present invention provides a computer device comprising a memory and a processor, the memory storing a computer program which, when executed by the processor, implements the steps of the method described in the first example;
and/or, when the computer program being executed by the processor, implement the steps of the method described in the second example;
and/or, when the computer program being executed by the processor, implement the steps of the method described in the third example;

In a sixth example of the present invention, the present invention provides a computer readable storage medium having stored thereon computer program instructions which, when executed by a processor, implement the steps of the method described in the first example;
and/or, when the program instructions being executed by the processor, implement the steps of the method described in the second example;
and/or, when the program instructions being executed by the processor, implement the steps of the method described in the third example.

The examples of the device described above are only schematic, wherein the units described as illustrated as separate components may or may not be physically separated, and the components displayed as units may or may not be physical units i.e. may be located in one place or may be distributed to a plurality of network units. Some or all of these modules can be selected according to practical needs to achieve the purpose of this embodiment. It can be understood and implemented by a person of ordinary skill in art without creative labor.

Through the description of the above embodiments, it is clear to those of ordinary skill in the art that each embodiment can be implemented by means of adding the necessary common hardware platform, or, of course, by means of a combination of hardware and software. Based on such an understanding, the above technical solutions that essentially or contribute to the prior art may be embodied in the form of a computer product, and the present invention may take the form of a computer program product implemented on one or more computer usable storage media containing computer usable program code therein, including but not limited to a disk memory, CD-ROM, optical memory, etc.

The foregoing is only a preferred example of the present application and is not intended to limit the present application. Although the present application is described in detail with reference to the foregoing examples, it is still possible for a person skilled in the art to modify the technical solutions documented in the foregoing embodiments or to make equivalent substitutions for some of the technical features thereof. Any modification, equivalent substitution, improvement, etc. made within the spirit and principles of this application shall be included in the scope of protection of this application.

1. A method for predicting disease risk based on multimodal fusion, comprising:
obtaining electronic health record (EHR) data of a patient, comprising structured data and unstructured data;
inputting the EHR data into a disease risk prediction model to obtain a disease risk prediction result; and
outputting the disease risk prediction result;
wherein, the disease risk prediction model performing steps comprising:
extracting structured data features and unstructured data features;
fusing the structured data features and the unstructured data features, and extracting fusion features; and
decision-making on the fusion features to obtain the disease risk prediction result.

2. The method as claimed in claim 1, wherein, using a Fully Convolutional Network (FCN) to extract the structured data features; and
using a Bidirectional Encoder Representation from Transformer (BERT) to extract the unstructured features.

3. The method as claimed in claim 1, wherein, an operation of extracting the fusion features comprises: connecting the unstructured data features and the structured data features in parallel along a specified dimension, reducing an imbalance rate through a method of analyzing minority class sample data and newly generating a sample of the class by using Synthetic Minority Oversampling Technique (SMOTE), then extracting the fusion features by using a piecewise pooling operation;
during the prediction, inputting the fusion features as an input into a fully connected dence layer, and then performing the prediction of disease risk by a Softmax classifier;
adopting a weighting of a cross-entropy loss and a hinge loss to jointly constrain the disease risk prediction model.

4. The method as claimed in claim 1, wherein, the prediction model of disease risk further comprises a step of performing a data cleaning before extracting the structured data features and the unstructured data features;
the data cleaning comprises replacing outlier values, completing missing values using mean values, and removing dirty read; and
the unstructured data is a text.

5. A disease risk prediction system based on multimodal fusion, comprising:
a feature extraction module, for extracting features on EHR data to obtain unstructured data features and structured data features;
a feature fusion module, for fusing the unstructured data features and the structured data features to extract and obtain fusion features; and
a classification module, for obtaining a disease risk prediction result by using the extracted fusion features as an input.

6. The system as claimed in claim 5, wherein, the feature extraction module comprises a structured data feature extraction module and an unstructured data feature extraction module;
wherein, the structured data feature extraction module uses a pre-processed structured data as an input of an FCN, maps the data to each hidden semantic node, and obtains the structured data features;
wherein, the unstructured data feature extraction module uses a BERT to extract features of the unstructured data; the BERT comprises a BERT Encoder comprising multiple BERT Layers, and each the BERT Layer is an Encoder Block in a Transformer; each the Encoder Block comprises two layers being a self-attentive mechanism layer and a feed-forward neural network layer, separately;
the feature fusion module connects the unstructured data features and the structured data features in parallel along a specified dimension, reduces an imbalance rate through a method of analyzing minority class sample data and newly generating the sample of the class by using a SMOTE, and then extracts the fusion features by using a piecewise pooling operation;
the classification module inputs the fusion features or the structured data as an input into a fully connected dence layer, and then predicts an outcome of a patient through a Softmax classifier;
the system further comprises a data acquisition module for obtaining the EHR data;
the system further comprises a data cleaning module for preprocessing the EHR data after obtaining the EHR data and before performing the feature extraction on the EHR data; wherein, the preprocessing comprises the EHR data cleaning module performing operations of replacing outlier values, completing missing values using mean values, and removing dirty read and
the system further comprises a result output module for outputting the prediction results of disease risk.

7. A method for processing EHR data, comprising: obtaining EHR data, the EHR data comprising structured data and unstructured data; performing data processing on structured data and unstructured data separately, including performing data cleaning to obtain cleaned structured data and cleaned unstructured data, performing feature extraction to obtain unstructured data features and structured data features, fusing unstructured data features and structured data features, and extracting fused features; and
using the fused features as data to be identified for medical purposes;
the data cleaning comprises replacing outlier values, completing missing values using mean values, and removing dirty read; and the unstructured data is text;
an FCN is used for extracting structured data features;
a BERT is used for extracting unstructured features;
an operation of extracting the fusion features comprises: connecting the unstructured data features and the structured data features in parallel along a specified dimension, reducing an imbalance rate through a method of analyzing minority class sample data, and newly generating the sample of the class by using a SMOTE, and then extracting fusion features by using a piecewise pooling operation.

8. A method of constructing a disease risk prediction model of the present invention, comprising:
obtaining EHR data of a patient with a known disease risk outcome, the data comprising structured data and unstructured data; building a dataset based on the obtained EHR data, the dataset comprises a structured dataset and an unstructured dataset; and building a label set based on a known outcome;
building a disease risk prediction network, comprising: building a feature extraction module for extracting features of the structured data, a feature extraction module for extracting features of the unstructured data and a feature fusion module, then connecting the structured data feature extraction module and the feature extraction module unstructured data in parallel and then being connected with the feature fusion module in series at a decision layer; the disease risk prediction network is implemented based on a Pytorch framework; and
training the disease risk prediction network using the datasets (the structured dataset and the unstructured dataset) with the label set as a label to build the disease risk prediction model;
before the dataset being built, further comprising a step of performing the data cleaning on the obtained EHR data, wherein the data cleaning comprises replacing outlier values, completing missing values using mean values, and removing dirty read;
the structured data feature extraction module is an FCN module;
the unstructured data feature extraction module is a BERT module;
the feature fusion module performs the steps of: connecting the unstructured data features and the structured data features in parallel along a specified dimension, reducing an imbalance rate through a method of analyzing minority class sample data and newly generating samples of the class by using a SMOTE, and then extracting the fusion features by using a piecewise pooling operation;
when using the dataset for training, inputting the fusion features, as an input, into a fully connected dence layer to train a Softmax classifier.

9. A computer device, comprising a memory and a processor, the memory storing a computer program, wherein, when the computer program being executed by the processor, implement steps of a method as claimed in claim 1;
and/or, when the computer program being executed by the processor, implement steps of the method for processing EHR data, comprising:
obtaining EHR data, the EHR data comprising structured data and unstructured data;
performing data processing on structured data and unstructured data separately, including performing data cleaning to obtain cleaned structured data and cleaned unstructured data, performing feature extraction to obtain unstructured data features and structured data features, fusing unstructured data features and structured data features, and extracting fused features; and
using the fused features as data to be identified for medical purposes; the data cleaning comprises replacing outlier values, completing missing values using mean values, and removing dirty read; and the unstructured data is text;
the FCN is used for extracting structured data features;
the BERT is used for extracting unstructured features;
the operation of extracting the fusion features comprises: connecting the unstructured data features and the structured data features in parallel along the specified dimension, reducing the imbalance rate through the method of analyzing minority class sample data, and newly generating the sample of the class by using the SMOTE, and then extracting fusion features by using the piecewise pooling operation
and/or, when the computer program being executed by the processor, implement steps of the method of constructing the disease risk prediction model of the present invention, comprising:
obtaining EHR data of the patient with the known disease risk outcome, the data comprising structured data and unstructured data; building the dataset based on the obtained EHR data, the dataset comprises the structured dataset and the unstructured dataset; and building the label set based on the known outcome;
building the disease risk prediction network, comprising: building the feature extraction module for extracting features of the structured data, the feature extraction module for extracting features of the unstructured data and the feature fusion module, then connecting the structured data feature extraction module and the feature extraction module unstructured data in parallel and then being connected with the feature fusion module in series at the decision layer; the disease risk prediction network is implemented based on the Pytorch framework; and
training the disease risk prediction network using the datasets (the structured dataset and the unstructured dataset) with the label set as the label to build the disease risk prediction model;
before the dataset being built, further comprising the step of performing the data cleaning on the obtained EHR data, wherein the data cleaning comprises replacing outlier values, completing missing values using mean values, and removing dirty read;
the structured data feature extraction module is the FCN module;
the unstructured data feature extraction module is the BERT module;
wherein, the feature fusion module performs the steps of: connecting the unstructured data features and the structured data features in parallel along the specified dimension, reducing the imbalance rate through the method of analyzing minority class sample data and newly generating samples of the class by using the SMOTE, and then extracting the fusion features by using the piecewise pooling operation; and
when using the dataset for training, inputting the fusion features, as the input, into the fully connected dence layer to train the Softmax classifier.

10. A computer readable storage medium having stored thereon computer program instructions, wherein, when the computer program instructions being executed by a processor, implement steps of a method as claimed in claim 1;
and/or, when the computer program instructions being executed by the processor, implement steps of the method for processing EHR data, comprising:
obtaining EHR data, the EHR data comprising structured data and unstructured data;
performing data processing on structured data and unstructured data separately, including performing data cleaning to obtain cleaned structured data and cleaned unstructured data, performing feature extraction to obtain unstructured data features and structured data features, fusing unstructured data features and structured data features, and extracting fused features; and
using the fused features as data to be identified for medical purposes;
the data cleaning comprises replacing outlier values, completing missing values using mean values, and removing dirty read; the unstructured data is text;
the FCN is used for extracting structured data features;
the BERT is used for extracting unstructured features;
the operation of extracting the fusion features comprises: connecting the unstructured data features and the structured data features in parallel along the specified dimension, reducing the imbalance rate through the method of analyzing minority class sample data, and newly generating the sample of the class by using the SMOTE, and then extracting fusion features by using the piecewise pooling operation
and/or, when the computer program instructions being executed by the processor, implement steps of the method of constructing the disease risk prediction model of the present invention, comprising:
obtaining EHR data of the patient with the known disease risk outcome, the data comprising structured data and unstructured data; building the dataset based on the obtained EHR data, the dataset comprises the structured dataset and the unstructured dataset; and building the label set based on the known outcome;
building the disease risk prediction network, comprising: building the feature extraction module for extracting features of the structured data, the feature extraction module for extracting features of the unstructured data and the feature fusion module, then connecting the structured data feature extraction module and the feature extraction module unstructured data in parallel and then being connected with the feature fusion module in series at the decision layer; the disease risk prediction network is implemented based on the Pytorch framework; and
training the disease risk prediction network using the datasets (the structured dataset and the unstructured dataset) with the label set as the label to build the disease risk prediction model;
before the dataset being built, further comprising the step of performing the data cleaning on the obtained EHR data, wherein the data cleaning comprises replacing outlier values, completing missing values using mean values, and removing dirty read;
the structured data feature extraction module is the FCN module;
the unstructured data feature extraction module is the BERT module;
the feature fusion module performs the steps of: connecting the unstructured data features and the structured data features in parallel along the specified dimension, reducing the imbalance rate through the method of analyzing minority class sample data and newly generating samples of the class by using the SMOTE, and then extracting the fusion features by using the piecewise pooling operation; and
when using the dataset for training, inputting the fusion features, as the input, into the fully connected dence layer to train the Softmax classifier.
